(19) United States
(12) Patent Application Publication
(10) Pub. No.: US 2025/0311998 A1
(43) Pub. Date: Oct. 9, 2025

(54) SYSTEMS AND METHODS FOR INTELLIGENT TELEMEDICINE

(71) Applicant: Chudi Adi, Albuquerque, NM (US)

(72) Inventor: Chudi Adi, Albuquerque, NM (US)

(21) Appl. No.: 18/629,805

(22) Filed: Apr. 8, 2024

Related U.S. Application Data
(60) Provisional application No. 63/573,949, filed on Apr. 3, 2024.

(51) Int. Cl. A61B 7/04; A61B 5/00; G16H 10/60; G16H 80/00

(52) U.S. Cl. CPC A61B 7/04; A61B 5/0004; A61B 5/0022; A61B 5/7425; G16H 10/60; G16H 80/00; A61B 2560/045

(57) ABSTRACT

A multimode stethoscope may provide an auscultatory signal from a patient to a local playback device and to an extended reality (XR) device. The multimode stethoscope may include a signal converter that converts the auscultatory signal to an electric signal carrying the auscultatory signal; an acoustic channel passing the auscultatory signal to an auscultation output port; a chest piece passing the auscultatory signal from the patient to the acoustic channel and to the signal converter; and a first wireless interface wirelessly transmitting the electric signal to the XR device. The auscultation output port may removably attach a stethoscope tube to the acoustic channel. The XR device may display an auscultation position on the patient's body wherein the multimode stethoscope may obtain the auscultatory signal when the chest piece is at the auscultation position. The XR device may store auscultation position data indicating the auscultation position in the patient's profile.

### CROSS REFERENCE TO RELATED APPLICATIONS

This patent application and claims the priority and benefit of U.S. provisional patent application No. 63/573,949, titled “EziScope Digital Stethoscope,” filed on Apr. 3, 2024, which is herein incorporated by reference in its entirety.

### TECHNICAL FIELD

The systems and methods relate to stethoscopes for telemedicine, to extended reality devices, to extended reality devices connected to multimode stethoscopes, and to relaying an auscultatory signal to a remote clinician. The systems and methods also relate to displaying an auscultation position on the body of a patient such that a multimode stethoscope may acquire the auscultatory signal when the multimode stethoscope is positioned at the auscultation position.

### BACKGROUND

Clinicians have long used stethoscopes to listen to the auscultatory signals produced by a patient's body. The auscultatory signals are best acquired when the chest piece of the stethoscope is positioned at specific auscultation positions on the body of a patient. For example, a clinician examining heart function may move the chest piece to one specific auscultation position, then another and then another to listen to different heart sounds. It takes considerable training and experience for a clinician to identify the proper auscultation positions because the positions may vary based on the patient's height, width, weight, etc. Other auscultation positions may be appropriate for examining lung function, bowel function, arterial function, etc.

Remote medicine and telemedicine may require a clinician to examine patients who are distant and for whom no trained personnel are available are near. Although stethoscopes intended for telemedicine exist, it has proven difficult to use auscultatory signals to diagnose bodily function in telemedicine settings because patients and assistants with the patients rarely have the level of training needed for placing a stethoscope chest piece at the correct auscultation positions.

### BRIEF SUMMARY OF SOME EXAMPLES

The following presents a summary of one or more aspects of the present disclosure to provide a basic understanding of such aspects. This summary is not an extensive overview of all contemplated features of the disclosure and is intended neither to identify key or critical elements of all aspects of the disclosure nor to delineate the scope of any or all aspects of the disclosure. Its sole purpose is to present some concepts of one or more aspects of the disclosure as a prelude to the more detailed description that is presented later.

An aspect of the subject matter described in this disclosure may be implemented by a system. The system may include a multimode stethoscope configured to provide an auscultatory signal from a patient to a local playback device and to an extended reality (XR) device, the multimode stethoscope comprising: a signal converter configured to convert the auscultatory signal to an electric signal that carries the auscultatory signal, an acoustic channel configured to pass the auscultatory signal to an auscultation output port, a chest piece configured to pass the auscultatory signal from the patient to the acoustic channel and to the signal converter, and a first wireless interface configured to wirelessly connect the multimode stethoscope to the XR device and to transmit the electric signal to the XR device, wherein the auscultation output port is configured to removably attach a stethoscope tube to the acoustic channel.

Another aspect of the subject matter described in this disclosure may be implemented with a non-transitory computer-readable storage medium comprising instructions that, when executed by an extended reality (XR) device, cause the XR device to wirelessly connect to a multimode stethoscope configured to provide an auscultatory signal from a patient to the XR device, display an auscultation position on a body of the patient, and acquire the auscultatory signal while a chest piece of the multimode stethoscope is positioned at the auscultation position.

Yet another aspect of the subject matter described in this disclosure may be implemented as a method. The method may include wirelessly connecting an extended reality (XR) device to a multimode stethoscope configured to provide an auscultatory signal from a patient to the XR device, acquiring an image of the patient, displaying an auscultation position on the image of a body of the patient in response to calculating the auscultation position from the image of the patient, and acquiring the auscultatory signal while a chest piece of the multimode stethoscope is positioned at the auscultation position.

In some implementations of the methods and devices, the system may further include the stethoscope tube and a stethoscope earpiece, the stethoscope earpiece configured to pass the auscultatory signal into an ear of a local listener. In some implementations of the methods and devices, the multimode stethoscope further includes a second wireless interface configured to wirelessly connect the multimode stethoscope to a local playback device and to transmit the electric signal to the local playback device. In some implementations of the methods and devices, the system may further include a non-transitory computer-readable storage medium comprising instructions that, when executed by the XR device, cause the XR device to display an auscultation position on a body of the patient, wherein the auscultatory signal is obtained from the patient by placing the chest piece at the auscultation position. In some implementations of the methods and devices, the auscultation position is displayed to a local assistant. In some implementations of the methods and devices, the auscultation position is displayed to the patient. In some implementations of the methods and devices, the instructions, when executed by the XR device, cause the XR device to connect the XR device to a remote computer, and display the auscultation position on the body of the patient in response to receiving the auscultation position from the remote computer. In some implementations of the methods and devices, the instructions, when executed by the XR device, cause the XR device to acquire an image of the patient, and display the auscultation position on the body of the patient in response to calculating the auscultation position from the image of the patient.

In some implementations of the methods and devices, the instructions, when executed by the XR device, cause the XR device to connect the XR device to a remote computer, and send the auscultatory signal to a remote clinician via the remote computer. In some implementations of the methods and devices, the instructions, when executed by the XR device, cause the XR device to relay a communication from the remote clinician to at least one of the patient and a local assistant, wherein the communication includes graphics displayed by the XR device. In some implementations of the methods and devices, the instructions, when executed by the XR device, cause the XR device to store a profile of the patient, the profile including auscultation position data that indicates the auscultation position relative to a plurality of physiological markers of the body of the patient. In some implementations of the methods and devices, the instructions, when executed by the XR device, cause the XR device to record a first auscultatory signal while the chest piece is at a first position, record a second auscultatory signal while the chest piece is at a second position, and update the auscultation position indicator to indicate a new auscultation position that is calculated from the first auscultatory signal and the second auscultatory signal. In some implementations of the methods and devices, the instructions, when executed by the XR device, cause the XR device to update a remotely stored patient profile of the patient in response to calculating the new auscultation position.

In some implementations, the instructions, when executed by the XR device, cause the XR device to acquire an image of the patient, and display the auscultation position on the body of the patient in response to calculating the auscultation position from the image of the patient. In some implementations, the instructions, when executed by the XR device, cause the XR device to connect the XR device to a remote computer, and send the auscultatory signal to a remote clinician via the remote computer. In some implementations, the instructions, when executed by the XR device, cause the XR device to store a profile of the patient, the profile including auscultation position data that indicates the auscultation position relative to a plurality of physiological markers of the body of the patient. In some implementations, the instructions, when executed by the XR device, cause the XR device to record a first auscultatory signal while the chest piece is at a first position, record a second auscultatory signal while the chest piece is at a second position, and update the auscultation position indicator to indicate a new auscultation position that is calculated from the first auscultatory signal and the second auscultatory signal.

In some implementations of the methods and devices, the method may further include storing a profile of the patient, the profile including auscultation position data that indicates the auscultation position relative to a plurality of physiological markers of the body of the patient, recording a first auscultatory signal while the chest piece is at a first position, recording a second auscultatory signal while the chest piece is at a second position, and updating the auscultation position indicator to indicate a new auscultation position that is calculated from the first auscultatory signal and the second auscultatory signal.

These and other aspects will become more fully understood upon a review of the detailed description, which follows. Other aspects and features will become apparent to those of ordinary skill in the art, upon reviewing the following description of specific examples in conjunction with the accompanying figures. While features may be discussed relative to certain examples and figures below, any example may include one or more of the advantageous features discussed herein. In other words, while one or more examples may be discussed as having certain advantageous features, one or more of such features may also be used in accordance with the various examples discussed herein. In similar fashion, while the examples may be discussed below as devices, systems, or methods, the examples may be implemented in various devices, systems, and methods.

### BRIEF DESCRIPTION OF THE DRAWINGS

FIG. 1 is a high level conceptual diagram illustrating an example of a multimode stethoscope, according to some aspects.

FIG. 2 is a high level block diagram illustrating an example of a multimode stethoscope, according to some aspects.

FIG. 3 is a high-level block diagram illustrating an example of a computer that a user may use for interacting with a multimode stethoscope, according to some aspects.

FIG. 4 is a high-level block diagram illustrating an example of a software system according to some aspects.

FIG. 5 is a high level conceptual diagram illustrating aspects of determining and displaying auscultation positions, according to some aspects.

FIG. 6 is a high level conceptual diagram illustrating an assistant using an extended reality (XR) device that displays an auscultation position on an image of the body of a patient, according to some aspects.

FIG. 7 is a high level conceptual diagram illustrating an assistant placing a multimode stethoscope on a patient such that a clinician may listen to an auscultatory signal from the patient, according to some aspects.

FIG. 8 is a high level flow diagram illustrating a method for acquiring an auscultatory signal from a patient, according to some aspects.

FIG. 9 is a high level flow diagram illustrating a process for a remote clinician and a patient or a local assistant to share auscultatory signals and other data, according to some aspects.

FIG. 10 is a high-level block diagram illustrating an example of a patient profile, according to some aspects.

FIG. 11 is a high-level conceptual diagram illustrating an example of a first auscultation position and four offset auscultation positions, according to some aspects.

FIG. 12 is a high level flow diagram illustrating a process for updating an auscultation position, according to some aspects.

Throughout the description, similar reference numbers may be used to identify similar elements.

### DETAILED DESCRIPTION

It will be readily understood that the components of the examples as generally described herein and illustrated in the appended figures could be arranged and designed in a wide variety of different configurations. Thus, the following more detailed description of various examples, as represented in the figures, is not intended to limit the scope of the present disclosure, but is merely representative of various examples. While the various aspects of the examples are presented in drawings, the drawings are not necessarily drawn to scale unless specifically indicated.

Systems and methods that implement aspects may have various differing forms. The described systems and methods are to be considered in all respects only as illustrative and not restrictive. The scope of the claims is, therefore, indicated by the claims themselves rather than by this detailed description. All changes which come within the meaning and range of equivalency of the claims are to be embraced within their scope.

Reference throughout this specification to features, advantages, or similar language does not imply that any system or method implements every aspect that may be realized. Rather, language referring to the features and advantages is understood to mean that a specific feature, advantage, or characteristic described in an example may be implemented in or by at least one example. Thus, discussions of the features and advantages, and similar language, throughout this specification may, but do not necessarily, refer to the same example.

Furthermore, the described features, advantages, characteristics, and aspects may be combined in any suitable manner in one or more systems or methods. One skilled in the relevant art will recognize, considering the description herein, that one example may be practiced without one or more of the specific features or advantages of another example. In other instances, additional features and advantages may be recognized in one example that may not be present in all the examples.

Reference throughout this specification to “one example”, “an example”, or similar language means that a particular feature, structure, or characteristic described in connection with the indicated example may be included in at least one example. Thus, the phrases “in one example”, “in an example”, and similar language throughout this specification may, but do not necessarily, all refer to the same example.

Telemedicine is a field in which a remote clinician may examine a patient. Telemedicine has been growing in popularity due to numerous factors. One factor is that many areas of the world lack local clinicians. Another factor is the convenience of consulting a clinician without traveling to and waiting at a hospital or other care facility. Yet another factor is that the patient and the clinician are not exposed to the pathogens that are consistently introduced to care facilities by patients seeking in person treatment. Many diagnostic techniques, such as auscultation, require a trained and experienced clinician. Auscultation is the act of listening to the sounds a patient's body makes, such as the sounds made by the patient's heart, lungs, arteries, and abdomen. In most settings, a stethoscope chest piece is placed on the patient's body and an auscultation signal is passed from the patient's body to the clinician via the stethoscope. Proper auscultation requires accurate positioning of the chest piece at various auscultation positions. It has proven difficult for patients and assistants to accurately position stethoscope chest pieces in telemedicine scenarios.

A multimode stethoscope and an extended reality (XR) device may alleviate the issues being experienced with auscultation in telemedicine settings. The multimode stethoscope may have numerous operating modes such that the multimode stethoscope may be used in numerous settings that include in person, telemedicine, etc. In traditional mode, the multimode stethoscope may function as a traditional stethoscope by passing the auscultatory signal from the chest piece to a stethoscope earpiece via tubing. The multimode stethoscope may operate in traditional mode without electric power from batteries or some other electric power source. In local monitor mode, the multimode stethoscope may send the auscultatory signal to a local playback device such as a Bluetooth speaker. In connected mode, the multimode stethoscope may send the auscultatory signal to a user device such as a smartphone. The smartphone may send the auscultatory signal to the clinician who is remotely located.

The user device may be an XR device. In an example, an XR device may display graphics that overlay the imagery acquired by the XR device's camera. In another example, an XR device may display graphics on a transparent display such that the graphics appear on objects in the user's field of view. A smartphone, a tablet computer, or a headset that places display devices over the wearer's eyes may be an XR device. The XR device may be used to display auscultation positions on the patient's body. As such, the patient or an assistant may place the stethoscope accurately such that the multimode stethoscope acquires good quality auscultatory signals. The auscultatory signals may then be sent to the clinician, recorded for later analysis etc.

Different people may have different auscultation positions, which is one of the reasons training and experience may be required for finding the proper auscultation positions on various patients. The proper auscultation positions for a patient may be stored in a patient profile. A training process may be used to find the proper auscultation positions. In an example, the XR device may guide the patient or assistant to place the chest piece at a series of positions at which auscultatory signals may be acquired. The volumes of the acquired signals and the locations of the series of positions may be used to determine the auscultation position that is to be stored in the patient profile.

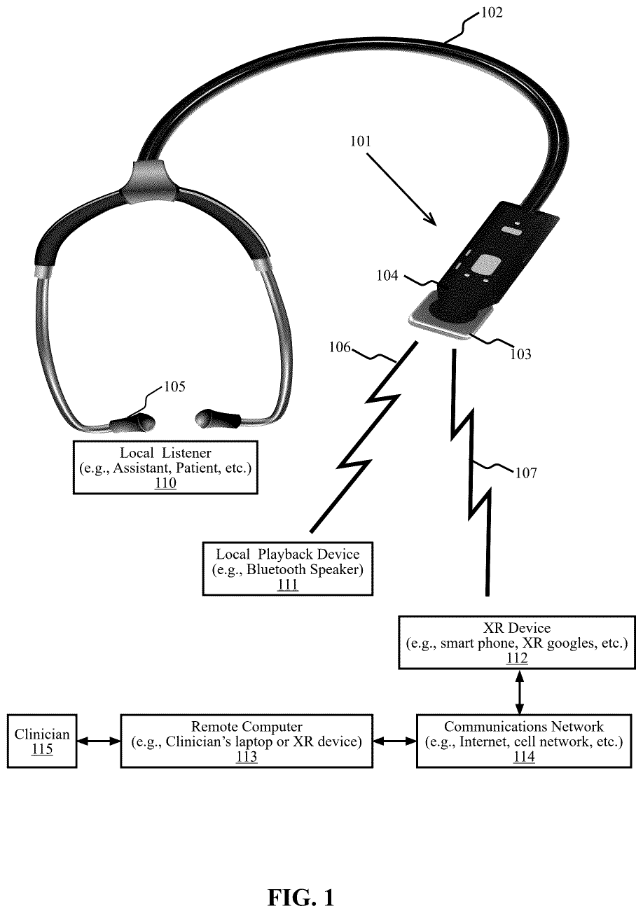

FIG. 1 is a high level conceptual diagram illustrating an example of a multimode stethoscope 101, according to some aspects. For brevity, the multimode stethoscope 101 may be referred to as the stethoscope 101. The stethoscope 101 may have a chest piece 103 and a housing 104. The chest piece 103 may be placed on the body of a patient to thereby receive an auscultatory signal from the patient. The auscultatory signal may pass from the chest piece into an acoustic channel in the housing. The acoustic channel in the housing may pass the auscultatory signal from the chest piece 103 to a stethoscope tube 102. The auscultatory signal may pass through the stethoscope tube 102 to a stethoscope earpiece 105 that may be positioned in the ear of a local listener 110 who may listen to the auscultatory signal.

The stethoscope 101 may include a signal converter that converts the auscultatory signal to an electric signal that carries the auscultatory signal. The electric signal may be sent via a wireless connection 106 to a local playback device 111 such as a Bluetooth speaker. The local playback device 111 may play the auscultatory signal such that the local listener 110 may listen to the auscultatory signal via the local playback device 111. As such, the stethoscope 101 may be operated without the stethoscope tube 102 attached. The electric signal may be sent via another wireless connection 107 to an XR device 112. The XR device may be connected to a remote computer 113 via a communications network 114 such as the internet. The remote computer 113 may be a laptop computer, a desktop computer, a handheld device, or another XR device being used by a clinician 115. The remote computer 113 may receive the auscultatory signal from the XR device 112 such that the clinician 115 may listen to the auscultatory signal. The remote computer may also send graphics (e.g., auscultation position indicators that the clinician places on an image of the patient's body), imagery (e.g., video of the clinician captured by the remote computer), text (e.g., text typed by the clinician), or audio (e.g., the clinician's voice) to the XR device 112 such that the clinician may thereby communicate with the patient or the assistant. The assistant is a person who may be present with and helping the patient. Here, the term “remote” does not indicate a specific physical distance. The term remote indicates that the clinician is using electronic means, such as the remote computer, to communicate with the patient or assistant. Similarly, the term “local” indicates that electronic means are not required for communications. For example, the assistant is local because the assistant may speak directly to the patient and may physically place the chest piece of the stethoscope on the patient.

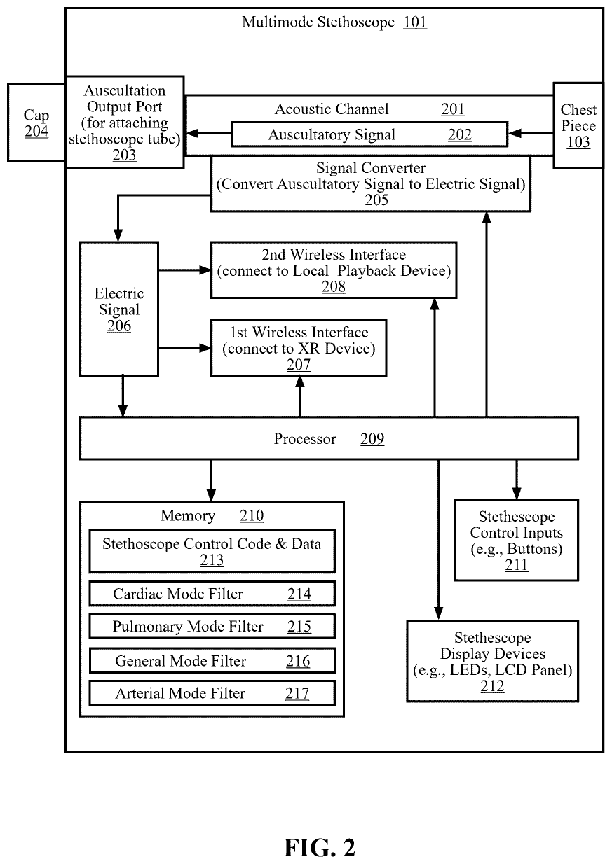

FIG. 2 is a high level block diagram illustrating an example of a multimode stethoscope 101, according to some aspects. The multimode stethoscope may include the chest piece 103, an acoustic channel 201, and an auscultation output port 203. The acoustic channel 201 may pass an auscultatory signal 202 from the chest piece 103 to the auscultation output port 203. A cap 204 covers the auscultation output port 203 such that debris does not enter the acoustic channel 201. The cap 204 may be removed and a stethoscope tube 102 may be attached to the auscultation output port 203 such that a local listener 110 may listen to the auscultatory signal 202 via a stethoscope earpiece 105. A signal converter 205 may be positioned on the acoustic channel 201 and may convert the auscultatory signal 202 to an electric signal 206. A first wireless interface 207 may receive the electric signal 206 and send the electric signal to a user device such as the XR device 112. A second wireless interface 208 may receive the electric signal 206 and send the electric signal to a local playback device 111.

The multimode stethoscope may include a memory 210 (e.g., a non-transitory memory) that stores stethoscope control code and data 213, a cardiac mode filter, a pulmonary mode filter 215, an arterial mode filter 217, and a general mode filter 216. The multimode stethoscope 101 may include a processor 209 that may run the stethoscope control code to thereby communicate via the wireless interfaces 207, 208, display information via the stethoscope display devices 212 (e.g., LEDs, LCD panels, etc.), and read stethoscope control inputs 211 (e.g., buttons, switches, knobs, sliders, capacitive sensors, etc.). The filters 214-217 may be the code and parameters for digital filters that the processor may implement. The different types of auscultatory signals have different characteristics. As such, filters have been developed that mask out unwanted sounds. A cardiac mode filter may mask out sounds that are not cardiac sounds. The pulmonary mode filter 215 may mask out sounds that are not lung sounds. The arterial mode filter 217 may mask out sounds that are not arterial sounds. The general mode filter 216 may mask out environmental sounds such as sounds that are not produced by a living body. Filtering auscultatory signals has been a subject of continuing research for generations. As such, filters published in the extant literature may be used.

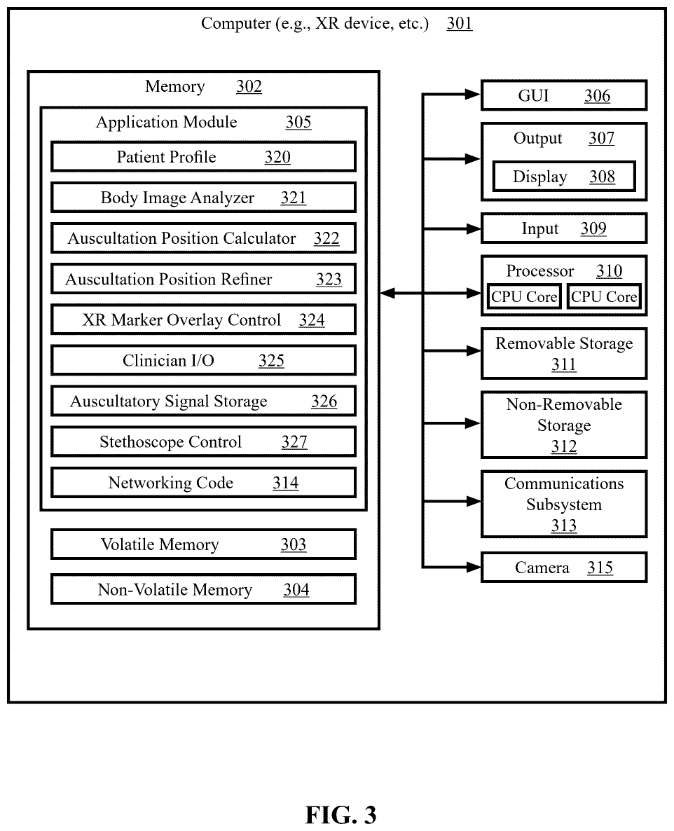

FIG. 3 is a high-level block diagram illustrating an example of a computer 301 that a user may use for interacting with a multimode stethoscope 101, according to some aspects. The XR device 112 may be implemented by a computer such as computer 301. A computing device in the form of a computer 301 configured to interface with controllers, peripheral devices, and other elements disclosed herein, may include one or more processors 310 coupled to memory 302, removable storage 311, and non-removable storage 312. Memory 302 may include volatile memory 303 and non-volatile memory 304. Computer 301 may include or have access to a computing environment that includes a variety of transitory and non-transitory computer-readable media such as volatile memory 303 and non-volatile memory 304, removable storage 311 and non-removable storage 312. Computer storage includes, for example, random access memory (RAM), read only memory (ROM), erasable programmable read-only memory (EPROM) and electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage, or other magnetic storage devices, or any other medium capable of storing computer-readable instructions as well as data. Of the listed computer storage, volatile memory, and most RAM, such as dynamic RAM (DRAM), are transitory while the others are considered non-transitory.

Computer 301 may include, or have access to, a computing environment that includes input 309, output 307, camera 315, and a communications subsystem 313. The computer 301 may operate in a networked environment using a communications subsystem 313 to connect to one or more remote computers, remote sensors and/or controllers, detection devices, hand-held devices, multi-function devices, speakers, mobile devices, tablet devices, mobile phones, smartphone, or other such devices. The communications subsystem may connect the computer 301 to a multimode stethoscope 101. The remote computer may also be a personal computer (PC), server, router, network PC, radio frequency identification (RFID) enabled device, a peer device, common network node, etc. The communications subsystem 313 may communicate via a communication connection that may include a local area network (LAN), a wide area network (WAN), Bluetooth connection, or other communication network.

Output 307 may be provided as a computer monitor, flat panel display, or transparent display but may include any output device. Output 307 and/or input 309 may include a data collection apparatus associated with computer 301. In addition, input 309, may include a typing input (e.g., keyboard) and/or pointing device (e.g., computer mouse, computer trackpad, touch screen, etc.) that allows a user to make selections or instruct the computer 301. A user interface can be provided using output 307 and input 309. Output 307 may include a display 308 for displaying data and information for a user, or for interactively displaying a graphical user interface (GUI) 306. A GUI is typically responsive to user inputs entered through input 309 and typically displays images and data on display 308. The camera 315 may acquire images that may be displayed via the GUI, analyzed by code in the application model, or displayed on the GUI with additional data such as auscultation position indicators overlaying imagery of a patient's body.

Note that the term “GUI” generally refers to a type of environment that represents programs, files, options, and so forth by means of graphically displayed icons, menus, and dialog boxes on a screen such as computer monitor or smartphone screen. In an example, a user may interact with the GUI to select and activate such options by directly touching the screen or pointing and clicking with a user input device 309 such as a pointing device (e.g., a mouse). A particular item can function in the same manner to the user in all applications because the GUI provides standard software routines (e.g., the application module 305 can include program code in executable instructions, including such software routines) to handle these elements and report the user's actions.

Computer-readable instructions, for example, program code in application module 305, may include or be representative of software routines, software subroutines, software objects, etc. that may be stored on a computer-readable medium and may be executable by the processor 310 of computer 301. The application module 305 may include computer code and data such as networking code 314, patient profile 320, body image analyzer 321, auscultation position calculator 322, auscultation position refiner 323, XR marker overlay control 324, clinician I/O 325, auscultatory signal storage 326, stethoscope control 327. A hard drive, CD-ROM, RAM, flash memory, and a USB drive are just some examples of articles including a computer-readable medium.

The networking code 314 and the communications subsystem may be used to communicate with the clinician's computer via the internet. The patient profile 320 may be data that specifically relates to the patient. The body image analyzer 321 may determine the locations of physiological markers on an image of a patient. The auscultation position calculator 322 may use the physiological markers to determine auscultation positions. The auscultation position refiner 323 may use data from several auscultatory signal readings to determine an auscultation position that may be stored in the patient profile. The XR marker overlay control 324 may overlay auscultation position indicators over imagery of a patient to thereby show auscultation positions on the body of the patient. Clinician I/O 325 may handle communications with a clinician via a remote computer. Auscultatory signal storage 326 may store previously acquired auscultatory signals. Stethoscope control 327 may send instructions to and receive status information from a multimode stethoscope.

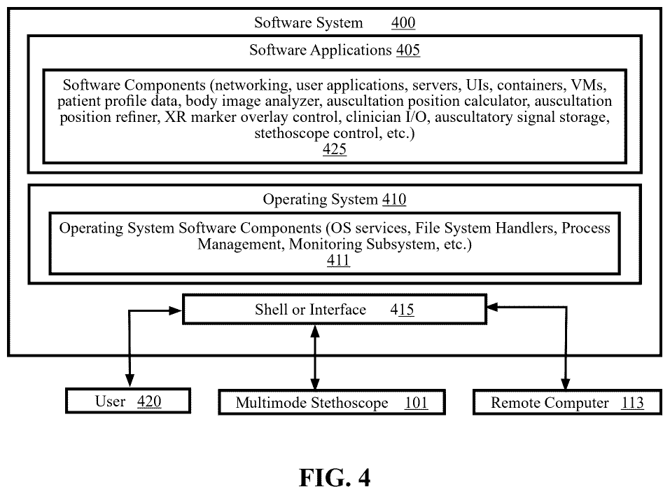

FIG. 4 is a high-level block diagram illustrating an example of a software system 400, according to some aspects. The software system 400 may be employed for directing the operation of data-processing systems such as computer 301. Software applications 405, may be stored in memory 302, on removable storage 311 or on non-removable storage 312, and generally includes and/or is associated with an operating system 410 and a shell or interface 415. One or more application programs may be “loaded” (i.e., transferred from removable storage 311 or non-removable storage 312 into the memory 302) for execution by the computer 301. Application programs 405 can include software components 425 such as software modules, software subroutines, software objects, network code, user application code, server code, UI code, container code, virtual machine (VM) code, patient profile data, body image analyzer, auscultation position calculator, auscultation position refiner, XR marker overlay control, clinician I/O, auscultatory signal storage, stethoscope control, etc. The software system 400 can have multiple software applications each containing software components. The computer 301 can receive user commands and data through interface 415, which can include input 309, output 307, and communications subsystem 313 accessible by a user 420, remote device such as remote computer 113, or a multimode stethoscope 101. These inputs may then be acted upon by the computer 301 in accordance with instructions from operating system 410 and/or software applications 405 and any software components 425 thereof. The operating system may include operating system software components 411 such as operating system services, file system handlers, process management, monitoring subsystem, etc.

Generally, software components 425 can include, but are not limited to, routines, subroutines, software applications, programs, modules, objects (used in object-oriented programs), executable instructions, data structures, etc., that perform specific tasks or implement specific abstract data types and instructions. Moreover, those skilled in the art will appreciate that elements of the disclosed methods and systems may be practiced with other computer system configurations such as, for example, XR devices, hand-held devices, mobile phones, smartphones, tablet devices, multi-processor systems, microcontrollers, printers, copiers, fax machines, multi-function devices, data networks, microprocessor-based or programmable consumer electronics, networked personal computers, minicomputers, mainframe computers, servers, medical equipment, medical devices, etc.

Note that the terms “component” and “module” as utilized herein may refer to one of or a collection of routines and data structures that perform a particular task or implement a particular abstract data type. Applications and components may be composed of two parts: an interface, which lists the constants, data types, variables, and routines that can be accessed by other modules or routines; and an implementation, which is typically private (accessible only from within the application or component) and which includes source code that implements the routines in the application or component. The terms application or component may also simply refer to an application such as a computer program designed to assist in the performance of a specific task such as word processing, accounting, etc. Components can be built or realized as special purpose hardware components designed to equivalently assist in the performance of a task.

The interface 415 can include a graphical user interface 306 that can display results, whereupon a user 420 or remote computer 113 may supply additional inputs or terminate a particular session. In some examples, operating system 410 and GUI 306 can be implemented in the context of a “windows” system. It can be appreciated, of course, that other types of systems are possible. For example, rather than a traditional “windows” system, other operating systems such as, for example, a real time operating system (RTOS) more commonly employed in wireless systems may also be employed with respect to operating system 410 and interface 415. The software application 405 can include, for example, software components 425 that may include instructions for carrying out steps or logical operations such as those shown and described herein.

The description herein is presented with respect to examples that may be implemented in the context of, or require the use of, a data processing system such as computer 301, in conjunction with program code in an application module 305 in memory 302, software system 400, or computer 301. The disclosed examples, however, are not limited to any specific application or environment. Instead, those skilled in the art will find that the systems and methods described herein may be advantageously applied to a variety of system and application software including database management systems, word processors, etc. Moreover, the examples may be implemented on a variety of different platforms including Windows, Macintosh, UNIX, LINUX, Android, Arduino, etc. Therefore, the descriptions of the examples which follow are for purposes of illustration and not considered a limitation.

Computers 301 and software systems 400 can take the form of or run as virtual machines (VMs) or containers that run on physical machines. A VM or container typically supplies an operating environment, appearing to be an operating system, to program code in an application module and software applications 405 running in the VM or container. A single physical computer can run a collection of VMs and containers. In fact, an entire network data processing system including a multitude of computers 301, LANs and perhaps even WANs or portions thereof can all be virtualized and running within a single computer (or a few computers) running VMs or containers. Those practiced in cloud computing are practiced in the use of VMs, containers, virtualized networks, and related technologies.

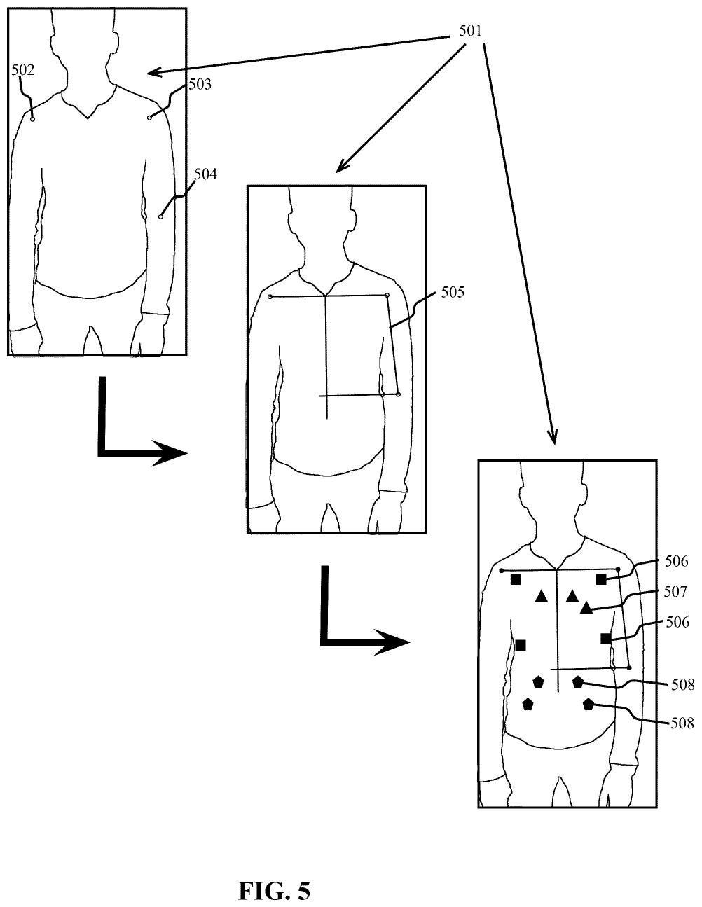

FIG. 5 is a high level conceptual diagram illustrating aspects of determining and displaying auscultation positions, according to some aspects. An image 501 of a patient may be acquired by a camera such as the camera 315 of an XR device. A body image analyzer 321 may analyze the image to identify physiological markers. A first physiological marker 502 may be the right shoulder joint. A second physiological marker 503 may be the left shoulder joint. A third physiological marker may be the left elbow joint. Clinicians may be trained to envision lines 505 defined by the physiological markers and to use those lines to locate auscultation positions. A first line may connect the shoulder joints. A second line may connect the left shoulder joint to the left elbow joint. A third line may bisect the first line and run along the center of the patient's body. A fourth line may be parallel to the first line and intersect the third physiological marker 504. A computer may also use the physiological markers to locate auscultation positions. For example, the first line may define a horizontal axis (the X axis) and the third line may define a vertical axis (the Y axis). The first physiological marker may be located at (Xa, Ya), the second physiological marker may be located at (Xb, Yb), and the third physiological marker may be located at (Xc, Yc). An auscultation position may be defined by two parameters (Px, Py). The location of the auscultation position on the image may be calculated as Xa+Px(Xb−Xa) along the X axis and Yb+Py(Yc−Yb) along the Y axis. For example, if Px=0.9 then the location of the auscultation position along the X axis may be 90% of the distance from (Xa, Ya) to (Xb, Yb). Similarly, if Py=0.6 then the location of the auscultation position along the Y axis may be 60% of the distance from (Xb, Yb) to (Xc, Yc). Certain pulmonary auscultation positions 506, cardiac auscultation positions 507, and abdominal auscultation positions 508 are indicated in FIG. 5. The auscultation positions 506, cardiac auscultation positions 507, and abdominal auscultation positions 508 may be calculated from the locations of the first physiological marker 502, the second physiological marker 503, and the third physiological marker 504.

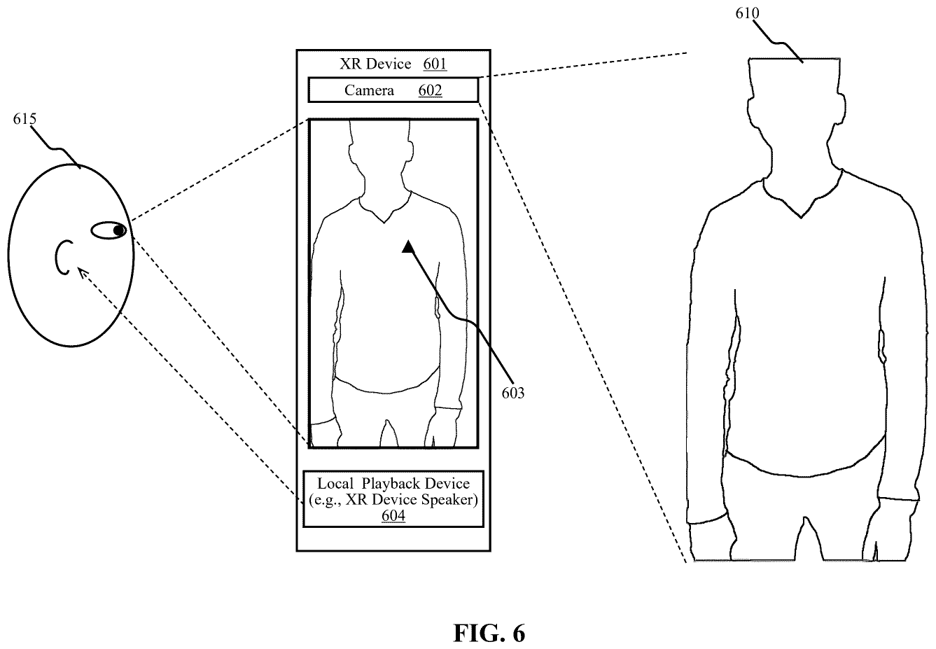

FIG. 6 is a high level conceptual diagram illustrating an assistant 615 using an XR device 601 that displays an auscultation position 603 overlaying an image of the body of a patient 610, according to some aspects. The XR device 601 may have a camera 602 and a speaker 604. The camera 602 may acquire an image of the patient and the XR device 601 may display the image to the assistant 615. Auscultation sounds acquired by a stethoscope may be played on the speaker 604 of the XR device 601 such that the assistant may hear the auscultation sounds. FIG. 6 illustrates a back facing camera acquiring the image of the patient. A back side camera on the back of the XR device may capture imagery that is shown on a display that is on the front side of the XR device. The XR device may include a front side camera. A front side camera on the front of the XR device may capture imagery that is shown on the display that is on the front side of the XR device. As such, the patient may use a front side camera to acquire the image of the patient's body and while observing the auscultation position 603 superimposed on or overlaying the image of the patient's body.

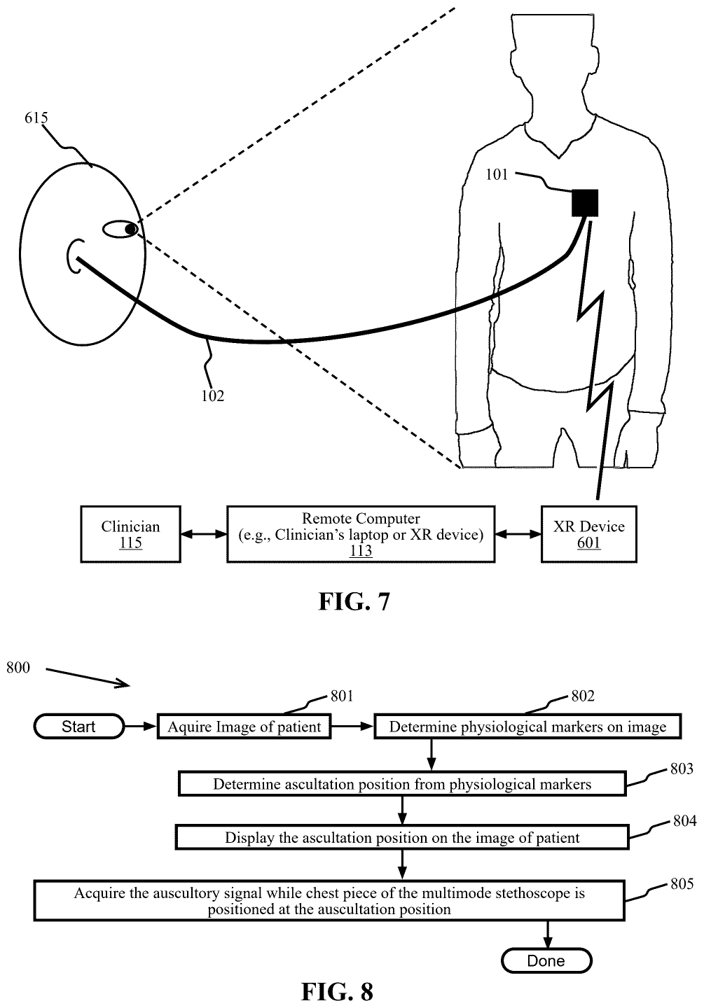

FIG. 7 is a high level conceptual diagram illustrating an assistant 615 placing a multimode stethoscope 101 on a patient such that a clinician 115 may listen to an auscultatory signal from the patient, according to some aspects. The multimode stethoscope 101 may wirelessly transmit the auscultatory signal to the XR device 601. The XR device may be connected to a remote computer 113 that the clinician 115 uses for listening to the auscultatory signal, for observing the patient, and for communicating with the patient and assistant 615. The remote computer may have a display that shows the image of the patient's body. The clinician may use a GUI of the remote computer to place graphics over the image of the patient's body. The graphic and the location for displaying the graphic may be transmitted from the remote computer 113 to the XR device 601 such that the assistant or patient may see the graphic on the image of the patient's body. In an example, the graphic is an auscultation position indicator. FIG. 7 illustrates the remote computer and the XR device relaying communications between the clinician 115 and the assistant 615 or the patient. A communication relayed from the clinician 115 to the assistant 615 may be video, audio, or graphics that the remote computer sends to the XR device. A communication relayed from the assistant 615 to the clinician 115 may be video, audio, or graphics that the XR device sends to the remote computer.

FIG. 8 is a high level flow diagram illustrating a method for acquiring an auscultatory signal from a patient 800, according to some aspects. The method illustrated in FIG. 8 may be performed by an XR device that communicates with a multimode stethoscope. After the start, an image of the patient may be acquired at block 801. At block 802, physiological markers are determined on the image. For example, the shoulders and the elbow joint may be identified as physiological markers. At block 803, auscultation positions are determined from the physiological markers. At block 804, the auscultation positions may be shown on the image of the patient. For example, auscultation position indicators may be overlaid over an image of the patient that is being displayed to an assistant or to the patient. At block 805, an auscultatory signal may be acquired while the chest piece of a multimode stethoscope is positioned at the auscultation position.

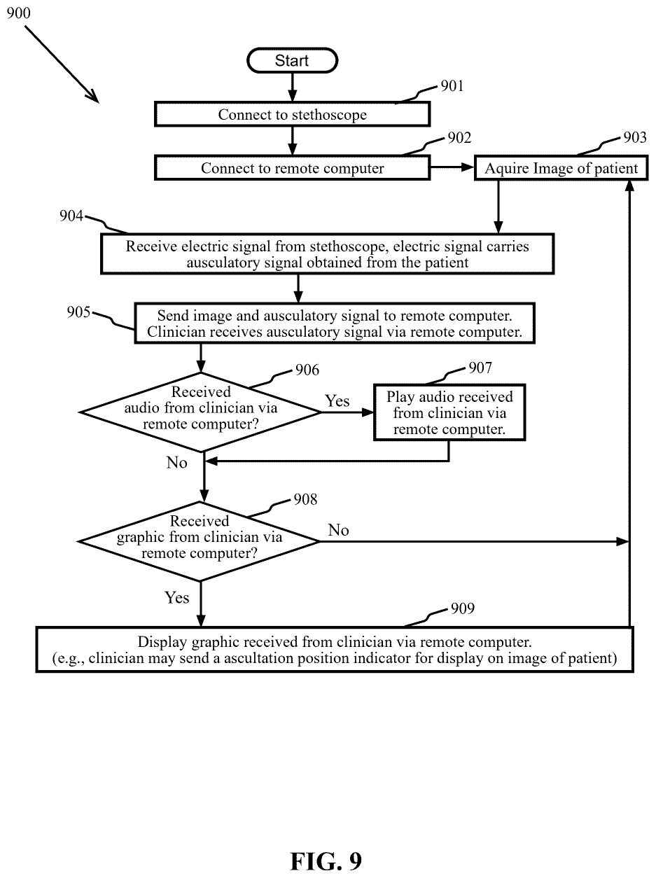

FIG. 9 is a high level flow diagram illustrating a process for a remote clinician and a patient or a local assistant to share auscultatory signals and other data 900, according to some aspects. The process of FIG. 9 may be performed by an XR device. After the start, a connection to a multimode stethoscope may be established at block 901. At block 902, a connection to a remote computer may be established. At block 903, an image of a patient may be acquired. In an example, the XR device includes a camera that acquires the image. At block 904, an electric signal may be received from the multimode stethoscope. The electric signal may carry an auscultatory signal acquired by the stethoscope from a patient. At block 905, the image and the auscultatory signal may be sent to a remote computer. A clinician may receive the image and the auscultatory signal via the remote computer. If audio (e.g., voice) from the clinician has been received by the XR device at decision block 906 then the process may move to block 907 and otherwise the process may move to decision block 908. At block 907, the audio received from the clinician may be played by the XR device. For example, a speaker in or connected to the XR device may play the audio. If a graphic (e.g., video of the clinician, a visual marker with a location at which to display to marker, etc.) from the clinician has been received by the XR device at decision block 908 then the process may move to block 909 and otherwise the process may loop back to block 903. At block 909, the graphic received from the clinician may be shown on a display of the XR device. For example, the XR device's display may display live video of the clinician or may display a graphic (e.g., an auscultation position indicator) received from the clinician on an image of the patient. From block 909 the process may loop back to block 903.

Clinicians have named various commonly used auscultation positions. For example, “V4” is one of the commonly used cardiac auscultation positions at which a cardiac auscultatory signal may be acquired. Trained clinicians may know the general location of an auscultation position such as V4, but might not know its exact position on a specific patient. A trained clinician can quickly adapt to a patient's physiology and thereby acquire the desired auscultatory signal. Untrained personnel may be incapable of such adaptation. As such, the auscultation position data in the patient profile may require refinement such that the auscultation positions for a patient are accurately indicated in that patient's patient profile.

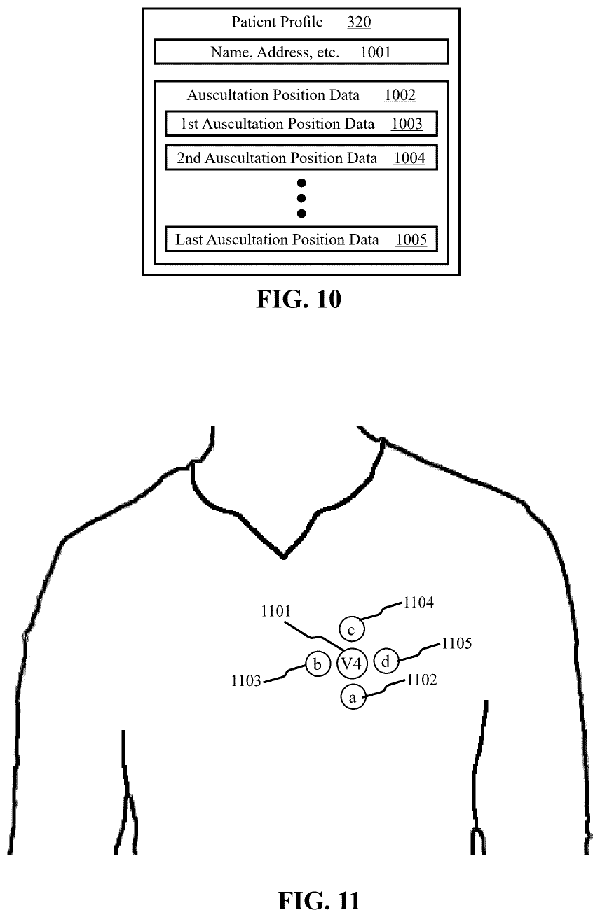

FIG. 10 is a high-level block diagram illustrating an example of a patient profile 320, according to some aspects. The patient profile 320 may include biographical data 1001 (e.g., name, address, etc.) and auscultation position data 1002 of the patient. The auscultation position data may include first auscultation position data 1003, second auscultation position data 1004, and last auscultation position data 1005. Auscultation position data may include values from which an auscultation position may be determined. For example, auscultation position data may include two parameters (Px, Py) that may be used to calculate the auscultation position from physiological markers. In an example, the first physiological marker may be located at (Xa, Ya), the second physiological marker may be located at (Xb, Yb), and the third physiological marker may be located at (Xc, Yc). The location of the auscultation position on the image may be calculated as Xa+Px(Xb−Xa) along the X axis and Yb+Py(Yc−Yb) along the Y axis. The auscultation positions are not the same for all patients due to differences in physiology. As such, each patient's patient profile may contain the patient's auscultation position data.

FIG. 11 is a high-level conceptual diagram illustrating an example of a first auscultation position 1101 and four offset auscultation positions, according to some aspects. The first auscultation position 1101 may be the initial V4 position. The second auscultation position 1102 may be an offset auscultation position that is below the first auscultation position 1101. The third auscultation position 1103 may be an offset auscultation position that is to the right (the patient's right) of the first auscultation position 1101. The fourth auscultation position 1104 may be an offset auscultation position that is above the first auscultation position 1101. The fifth auscultation position 1105 may be an offset auscultation position that is to the left (the patient's left) of the first auscultation position 1101. An auscultation position may be refined by acquiring auscultatory signals at the first auscultation position 1101, the second auscultation position 1102, the third auscultation position 1103, the fourth auscultation position 1104, and the fifth auscultation position 1105 and then using the acquired signals to determine an auscultation position to store in the patient's patient profile.

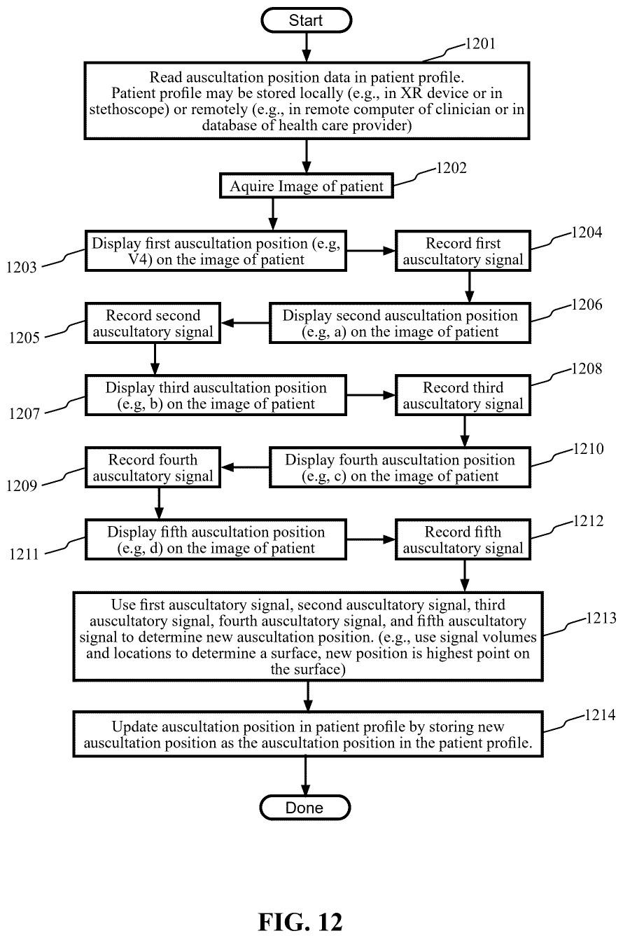

FIG. 12 is a high level flow diagram illustrating a process for updating an auscultation position, according to some aspects. After the start, the auscultation position data in the patient profile may be read at block 1201. The patient profile may be a locally stored patient profile (e.g., in the XR device or in the stethoscope) or a remotely stored patient profile (e.g., in the remote computer of the clinician or in a database of a healthcare provider). At block 1202, an image of the patient is acquired. At block 1203, the first auscultation position may be determined from the image and the auscultation position data. The first auscultation position may then be shown overlayed on the image of the patient. At block 1204, the first auscultatory signal may be acquired while the chest piece of the stethoscope is at the first auscultation position. At block 1205, the second auscultation position may be determined as an offset from the first auscultation position and then shown overlayed on the image of the patient. At block 1206, the second auscultatory signal may be acquired while the chest piece of the stethoscope is at the second auscultation position. At block 1207, the third auscultation position may be determined as an offset from the first auscultation position and then shown overlayed on the image of the patient. At block 1208, the third auscultatory signal may be acquired while the chest piece of the stethoscope is at the third auscultation position. At block 1209, the fourth auscultation position may be determined as an offset from the first auscultation position and then shown overlayed on the image of the patient. At block 1210, the fourth auscultatory signal may be acquired while the chest piece of the stethoscope is at the fourth auscultation position. At block 1211, the fifth auscultation position may be determined as an offset from the first auscultation position and then shown overlayed on the image of the patient. At block 1212, the fifth auscultatory signal may be acquired while the chest piece of the stethoscope is at the fifth auscultation position. At block 1213 the first auscultatory signal, the second auscultatory signal, the third auscultatory signal, the fourth auscultatory signal, and the fifth auscultatory signal may be used to determine a new auscultation position. For example, the location of an auscultation position may be interpreted as a grid location and the volume of the signal may be interpreted as a height at that grid location. The five measured signals at the five locations may therefore define a surface. The new auscultation position may be the highest point on the surface. At block 1214, the auscultation position in the patient's profile may be updated by storing the new auscultation position as the auscultation position in the patient profile.

Aspects described above may be ultimately implemented in a networking device that includes physical circuits that implement digital data processing, storage, and communications. The networking device may include processing circuits, read only memory (ROM), random access memory (RAM), ternary content-addressable memory (TCAM), and at least one interface (interface(s)). The CPU cores described above may be implemented in processing circuits and memory that may be integrated into the same integrated circuit (IC) device as ASIC circuits and memory that are used to implement the programmable packet processing pipeline. For example, the CPU and other semiconductor chip circuits are fabricated on the same semiconductor substrate to form a System-on-Chip (SoC). The networking device may be implemented as a single IC device (e.g., fabricated on a single substrate) or the networking device may be implemented as a system that includes multiple IC devices connected by, for example, a printed circuit board (PCB). The interfaces may include network interfaces (e.g., Ethernet interfaces and/or InfiniBand interfaces) and/or PCIe interfaces. The interfaces may also include other management and control interfaces such as inter-integrated circuit (I2C), general purpose input/outputs (IOs), universal serial bus (USB), universal asynchronous receiver/transmitter (UART), serial peripheral interface (SPI), and embedded multimedia card (eMMC).

Although the operations of the method(s) herein are shown and described in a particular order, the order of the operations of each method may be altered so that certain operations may be performed in an inverse order or so that certain operations may be performed, at least in part, concurrently with other operations. Instructions or sub-operations of distinct operations may be implemented in an intermittent and/or alternating manner.

It may also be noted that at least some of the operations for the methods described herein may be implemented using software instructions stored on a computer usable storage medium for execution by a computer. For example, a computer program product may include a computer usable storage medium to store a computer readable program.

The computer-usable or computer-readable storage medium may be an electronic, magnetic, optical, electromagnetic, infrared, or semiconductor system (or apparatus or device). Examples of non-transitory computer-usable and computer-readable storage media include a semiconductor or solid-state memory, magnetic tape, a removable computer diskette, a random-access memory (RAM), a read-only memory (ROM), a rigid magnetic disk, and an optical disk. Current examples of optical disks include a compact disk with read only memory (CD-ROM), a compact disk with read/write (CD-R/W), and a digital video disk (DVD).

Although specific examples have been described and illustrated, the scope of the claimed systems, methods, devices, etc. is not to be limited to the specific forms or arrangements of parts so described and illustrated. The scope may be defined by the claims appended hereto and their equivalents.

What is claimed is:

1. A system comprising:
a multimode stethoscope configured to provide an auscultatory signal from a patient to a local playback device and to an extended reality (XR) device, the multimode stethoscope comprising:
a signal converter configured to convert the auscultatory signal to an electric signal that carries the auscultatory signal;
an acoustic channel configured to pass the auscultatory signal to an auscultation output port;
a chest piece configured to pass the auscultatory signal from the patient to the acoustic channel and to the signal converter; and
a first wireless interface configured to wirelessly connect the multimode stethoscope to the XR device and to transmit the electric signal to the XR device,
wherein the auscultation output port is configured to removably attach a stethoscope tube to the acoustic channel.

2. The system of claim 1, further comprising the stethoscope tube and a stethoscope earpiece, the stethoscope earpiece configured to pass the auscultatory signal into an ear of a local listener.

3. The system of claim 1, wherein the multimode stethoscope further includes a second wireless interface configured to wirelessly connect the multimode stethoscope to a local playback device and to transmit the electric signal to the local playback device.

4. The system of claim 1, further comprising a non-transitory computer-readable storage medium comprising instructions that, when executed by the XR device, cause the XR device to:
display an auscultation position on a body of the patient,
wherein the auscultatory signal is obtained from the patient by placing the chest piece at the auscultation position.

5. The system of claim 4, wherein the auscultation position is displayed to a local assistant.

6. The system of claim 4, wherein the auscultation position is displayed to the patient.

7. The system of claim 4, wherein the instructions, when executed by the XR device, cause the XR device to:
connect the XR device to a remote computer; and
display the auscultation position on the body of the patient in response to receiving the auscultation position from the remote computer.

8. The system of claim 4, wherein the instructions, when executed by the XR device, cause the XR device to:
acquire an image of the patient; and
display the auscultation position on the body of the patient in response to calculating the auscultation position from the image of the patient.

9. The system of claim 4, wherein the instructions, when executed by the XR device, cause the XR device to:
connect the XR device to a remote computer; and
send the auscultatory signal to a remote clinician via the remote computer.

10. The system of claim 9, wherein the instructions, when executed by the XR device, cause the XR device to:
relay a communication from the remote clinician to at least one of the patient and a local assistant,
wherein the communication includes graphics displayed by the XR device.

11. The system of claim 4, wherein the instructions, when executed by the XR device, cause the XR device to:
store a profile of the patient, the profile including auscultation position data that indicates the auscultation position relative to a plurality of physiological markers of the body of the patient.

12. The system of claim 11 wherein the instructions, when executed by the XR device, cause the XR device to:
record a first auscultatory signal while the chest piece is at a first position;
record a second auscultatory signal while the chest piece is at a second position; and
update the auscultation position indicator to indicate a new auscultation position that is calculated from the first auscultatory signal and the second auscultatory signal.

13. The system of claim 12, wherein the instructions, when executed by the XR device, cause the XR device to update a remotely stored patient profile of the patient in response to calculating the new auscultation position.

14. A non-transitory computer-readable storage medium comprising instructions that, when executed by an extended reality (XR) device, cause the XR device to:
wirelessly connect to a multimode stethoscope configured to provide an auscultatory signal from a patient to the XR device;
display an auscultation position on a body of the patient; and
acquire the auscultatory signal while a chest piece of the multimode stethoscope is positioned at the auscultation position.

15. The non-transitory computer-readable storage medium of claim 14, wherein the instructions, when executed by the XR device, cause the XR device to:
acquire an image of the patient; and
display the auscultation position on the body of the patient in response to calculating the auscultation position from the image of the patient.

16. The non-transitory computer-readable storage medium of claim 14, wherein the instructions, when executed by the XR device, cause the XR device to:
connect the XR device to a remote computer; and
send the auscultatory signal to a remote clinician via the remote computer.

17. The non-transitory computer-readable storage medium of claim 14, wherein the instructions, when executed by the XR device, cause the XR device to:
store a profile of the patient, the profile including auscultation position data that indicates the auscultation position relative to a plurality of physiological markers of the body of the patient.

18. The non-transitory computer-readable storage medium of claim 17, wherein the instructions, when executed by the XR device, cause the XR device to:
record a first auscultatory signal while the chest piece is at a first position;
record a second auscultatory signal while the chest piece is at a second position; and
update the auscultation position indicator to indicate a new auscultation position that is calculated from the first auscultatory signal and the second auscultatory signal.

19. A method comprising:
wirelessly connecting an extended reality (XR) device to a multimode stethoscope configured to provide an auscultatory signal from a patient to the XR device;
acquiring an image of the patient;
displaying an auscultation position on the image of a body of the patient in response to calculating the auscultation position from the image of the patient; and
acquiring the auscultatory signal while a chest piece of the multimode stethoscope is positioned at the auscultation position.

20. The method of claim 19, further including:
storing a profile of the patient, the profile including auscultation position data that indicates the auscultation position relative to a plurality of physiological markers of the body of the patient;
recording a first auscultatory signal while the chest piece is at a first position;
recording a second auscultatory signal while the chest piece is at a second position; and
updating the auscultation position indicator to indicate a new auscultation position that is calculated from the first auscultatory signal and the second auscultatory signal.
